(19) United States
(12) Patent Application Publication
(10) Pub. No.: US 2004/0185543 A1
(43) Pub. Date: Sep. 23, 2004

(54) Process for production of ethanol using stable yeast crystals in modified conventional batch reactor

(75) Inventors: Shetty Prakasham Reddy, Hyderabad (IN); Ponnapalli Nageshwara Sarma, Hyderabad (IN); Srinivasulureddy Venkata Mohan, Hyderabad (IN); Katuri Krishna Prasad, Hyderabad (IN); Kondapuram Vijaya Raghavan, Hyderabad (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/393,402

(22) Filed: Mar. 20, 2003

(51) Int. Cl. C12P 7/06; C12N 1/18

(52) U.S. Cl. 435/161; 435/254.21

(57) ABSTRACT

Ethanol demand is ever increasing mainly because of its utilization as organic solvent and as starting compound for production of many organic solvents. In addition, it can replace the natural petroleum sources as energy fuels. Conventional method of ethanol production is performed mainly by batch fermentation, which requires addition of fresh yeast for every batch as seed culture and needs maintenance of yeast cultures, thus the process is costly and requires special expertise in microbiology. In this context, the recently developed yeast crystals solve some of the above mentioned problems. However, the developed biocatalytic crystals upon fermentation tend to float on the surface of the fermentation broth during fermentation and thereby increase the fermentation time and reduce the rate of alcohol fermentation. The novelty lying in the present invention is use of modified conventional batch reactor for fermentation with activated yeast crystals reduces the fermentation time drastically and enhances the rate of ethanol production.

### FIELD OF THE INVENTION

The present invention relates to an improved process for the production of ethanol using activated stable yeast crystals in modified conventional batch reactor. This invention more particularly relates to the reduction of fermentation time without compromising on the ethanol production in a batch process and improved the rate of ethanol production.

### BACKGROUND OF THE INVENTION

Ethanol is one of the largest volume chemicals produced biotechnologically since several decades. Its demand is ever increasing mainly due to the role of ethanol as a solvent, germicide, antifreezent and as chemical raw material for the production of various organic compounds such as acetaldehyde, acetic acid, butadiene and ethylene etc. Moreover, its importance as a valuable alternative source as transportation fuel has renewed interest in development of improved fermentation process to achieve higher ethanol yields.

Even today, ethanol production is carried out by conventional batch fermentation techniques using Sachhromyces cereviceae cultures. This process of ethanol fermentation with yeast requires maintenance of yeast cultures and pre-fermentation to get a required; quantity of yeast biomass for main fermentation. Hitherto several attempts have been made to enhance the ethanol production with free and immobilized yeast cells. The free cell fermentation is generally performed, using Saccharomyces cereviceae in diluted molasses solutions in a batch reactor for a period of 24-48 hours and requires development of yeast for every batch [S. V. Ramakrishna, V. P. Sreedharan and P. Prema. In: Bioreactor Immobilized Enzymes and Cells: Fundamentals and Applications (Ed Mood Young), Elsevier Appl. Sci., Amsterdam, 1988, 251-260]. Low productivity, higher fermentation time, higher operating and capital investment are some of the major disadvantages of the batch process [D. Das, R. G. Nandkishor, K. Murali and P. S. Gupta J. Ferment Bioengg. 1993, 75, 132-137; D. Weuster-Botz Appl. Microbiol. Biotechnol, 1993. 39. 679-684]. Many alternative fermentation strategies such as Boinot fermentation process [J. M. Lagomasino, International Sugar Journal, 1949, 51, 338-342], continuous floss fermentation [J. L. McCarthy, In: Industrial fermentations (Ed. L. A. Underkofler and R. J. Hickey). 1954, Vol.1, p 95; Chem. Pub. Co., New York], high cell densities in the fermentor, larger throughput by continuous mode of operation using cell recycle [C. W. Lee and H. N. Chang Biotehcnol. Bioengg, 1987, 29, 1105-1112], extractive fermentation [M. Minier and G. Goma, Biotehcnol. Bioengg. 1982, 24, 1565-1579] and whole cell immobilization by various techniques [S. V. Ramakrishna and R. S. Prakasham Current Science, 1999, 77, 87-100] have been attempted to enhance volumetric productivity of the system. But these methodologies suffer from the fact that the yeast cells has to be added in every batch. Continuous fermentation with cell recycles [E. J. DelRosario, K. J. Lee and P. L. Rogers. Biotechnology Bioengineering, 1979, 21, 1477; T. K. Ghosh and R. D. Tyagi Biotechnol. Bioengg, 1979, 21; 1387; G. H. Gil, W. J. Jones and T. G. Tornabene Enzyme Microb. Technol., 1991, 13; 390] and vacuume fermentation [G. R. Cysewski and C. R. Wilke Biotechnol Bioengg, 1977, 19; 1125] have resulted in substantial increase in the productivity. However, the cell recycling system involves considerable cost input for separation of yeast cells from the fermented broth. One of the attractive alternate method received wide attention is the high cell density fermentation. In this regard the use of immobilized yeast cells, instead of free cells. has been attempted by several researchers [R. Jamuna and S. V. Ramakrishna Biomass Bioenergy, 1992, 3; 117-119]. Various types of reactor configurations have been attempted to produce ethanol with entrapped cells [F. Godia, C. Casad, and C. Sola Process Biochem, 1987, 43-48]. One of the major limitations with entrapped cells is that the gas generated during fermentation get entrapped in the gel particles which in turn lowers the density and disintegrates the beads [S. V. Ramakrishna, V. P. Sreedharan and P. Prema. In: Bioreactor Immobilized Enzymes and Cells: Fundamentals and Applications (Ed Mood Young). Elsevier Appl. Sci., Amsterdam, 1988, 251-260]. Recently, S. V. Ramakrishna et al (1999) have developed stable yeast crystals and showed that they can be recycled several time [S. V. Ramakrishna. R. S. Prakasham and P. Komariah, Indian Patent Application No. 186/DEL/2000; U.S. patent application Ser. No. 09/538181]. The major limitation with yeast crystals is that they tend to float on the surface of the fermentation broth during fermentation thereby decreasing the rate of ethanol production. The novelty lying in the present invention is the use of activated stable yeast crystals in a modified batch bioreactor, which reduces the fermentation time drastically and enhances the rate of ethanol production.

### OBJECTS OF THE INVENTION

The main object of the invention is to provide a process for the production of ethanol using novel activated clustered yeast crystals in a modified conventional batch reactor.

Another object of the invention is to enhance the rate of ethanol production.

Yet another object of the invention is to improve the existing distillery production capacity more than 250% without expansion and compromising on the ethanol yield.

Still another object of the invention is to provide a cost effective process for the production of ethanol.

Still another object of the invention is to reduce the fermentation time.

### SUMMARY OF THE INVENTION

The novelty in the present invention resides inter alia, in the reduction of fermentation time from 28-36 hours to 8-16 hours by use of activated stable yeast crystals in a modified conventional batch reactor and in the considerable improvement on the rate of ethanol production. The present invention also provides improvement of existing distillery capacity to the tune of 250% without much machinery expansion and improved alcohol yield per tone of molasses used compared to conventional free cell fermentation.

Accordingly, the present invention provides a process for production of ethanol using activated stable yeast crystals in a modified conventional batch reactor, said process comprising mixing stable yeast crystals to a low strength molasses solution having specific gravity in the range of 1.030 to 1.060 and incubating the stable yeast crystals therein to obtain activated stable yeast crystals, separating the activated stable yeast crystals, converting the low strength molasses solution into a molasses fermentation broth of a specific gravity in the range of 1.09 to 1.1 in a modified fermentation vessel fitting with a low speed mechanical stirrer, estimating the initial sugar concentration in the fermentation broth, adding the activated stable yeast crystals to the above fermentation broth in the range of 0.5 to 2.0% W/V and fermenting the mixture, terminating the fermentation when the specific gravity of the fermentation broth reaches a range of 1.014-1.045, separating the activated yeast crystals from fermented broth and recovering the ethanol from fermentation broth.

In one embodiment of the invention, the incubation of the stable yeast crystals in the low strength molasses solution is carried out for a period of 4-48 hours at a temperature ranging between 24-36° C.

In another embodiment of the invention, the low strength molasses solution is obtained by mixing molasses with water.

In a further embodiment of the invention, fermentation is carried out for a time period in the range of 8-16 hours and at a temperature in the range of 28-40° C.

In another embodiment of the invention the activated stable yeast crystals added in fermentation broth is preferably in the range of 0.5-1.5% (W/V).

In still another embodiment of the present invention. the activated stable yeast crystals are separated from the molasses solution by draining out the solution or straining through a mesh or perforated bottoms.

In another embodiment of the invention, the low strength molasses solution of specific gravity in the range of 1.030 to 1.060 is prepared by mixing molasses and water by dilution or mechanical mixing.

In the present invention, stable yeast crystals were prepared as per the methodology given in our earlier patent [S. V. Ramakrishna, R. S. Prakasham and P. Komariah, Indian Patent Application No. 186/DEL/2000; U.S. patent application Ser. No. 09/538181].

### DETAILED DESCRIPTION OF THE INVENTION

The present invention of an improved ethanol production process using activated stable yeast crystals in modified conventional batch reactor is described with reference to the following examples which are explained by way of illustration only and should not therefore be construed to limit the scope of the present invention.

### EXAMPLE 1

### Ethanol Production Using Activated Yeast Crystals in Conventional Batch Reactor

Sugar cane molasses was procured from local sugar industry and stored at 4° C. till further use. The stable yeast crystals were prepared as described in the earlier patent [Indian patent application No. 186/DEL/2000 and U.S. patent application Ser. No. 09/538181] and activated by incubating in the 1.060 specific gravity molasses solution for 8 hours at 28° C.

Fermentation broth (100 liters) was prepared by diluting the molasses with water to get the specific gravity of the solution of 1.090 and the initial sugar concentration in the fermentation broth was measured using conventional procedures.

The activated stable yeast crystals were separated from activation solution and 1% of these crystals were added to conventional fermentation vessel. The fermentation was continued at 30° C. till the specific gravity of the fermentation broth reached to 1.024. The activated stable yeast crystals were then separated and the filtrate was used for ethanol recovery. The production of ethanol by using activated stable yeast crystals in conventional reactor is as follows.

TABLE 1

| S. NO. | PARAMETER | VALUES |
|---|---|---|
| 1. | Total volume (in liters) | 100.00 |
| 2. | Molasses used (Kg) | 28.20 |
| 3. | Initial specific gravity | 1.090 |
| 4. | Final specific gravity | 1.024 |
| 5. | Initial sugar concentration (%) | 14.68 |
| 6. | Sugar concentration in broth (%) | 1.86 |
| 7. | Alcohol produced (%) | 6.90 |
| 8. | Fermentation efficiency | 85.43 |
| 9. | Alcohol recovery per tone of molasses | 242.11 |
| 10. | Fermentation time | 30 |
| 11. | Rate of ethanol production | 8.07 |
| | (liters/hour/tone of molasses) | |

### EXAMPLE 2

### Ethanol Production Using Activated Yeast Crystals in Modified Conventional Reactor

The activated stable yeast crystals were recycled in this experiment. A low speed mechanical stirrer having rpm of 90 with hydrofoil impeller was fitted to conventional 1000-liter fermentation vessel. Fermentation broth (100 liters) was prepared by diluting the molasses with water to get the specific gravity of the solution of 1.090 and the initial sugar concentration in the fermentation broth was estimated using conventional procedures.

The activated stable yeast crystals (1%) were added to modified fermentation vessel. The fermentation was continued under constant mixing conditions in the modified fermentation vessel at 30° C. till the specific gravity of the fermentation broth was reached to 1.024. The activated stable yeast crystals were then separated and the filtrate was used for ethanol recovery. The production of ethanol by using activated stable yeast crystals in modified conventional reactor is as follows.

TABLE 2

| S. NO. | PARAMETER | VALUES |
|---|---|---|
| 1. | Total volume (in liters) | 100.00 |
| 2. | Molasses used (Kg) | 28.20 |
| 3. | Initial specific gravity | 1.090 |
| 4. | Final specific gravity | 1.024 |
| 5. | Initial sugar concentration (%) | 14.68 |
| 6. | Sugar concentration in broth (%) | 1.86 |
| 7. | Alcohol produced (%) | 7.04 |
| 8. | Fermentation efficiency | 87.10 |
| 9. | Alcohol recovery per tone of molasses | 247.02 |
| 10. | Fermentation time | 11 |
| 11. | Rate of ethanol production | 22.45 |
| | (liters/hour/tone of molasses) | |

### EXAMPLE 3

### Ethanol Production Using Activated Yeast Crystals in Conventional Batch Reactor

The same experiment was performed as trial 2. The activated stable yeast crystals of earlier experiments were reused in this experiment. Fermentation broth (75 liters) was prepared in conventional fermentation vessel by diluting the molasses with water to get the specific gravity of the solution of 1.090 and the initial sugar concentration in the fermentation broth was measured using conventional procedures.

One percent activated stable yeast crystals were added to fermentation broth. The fermentation was continued at 30° C. for 30 hours. The activated stable yeast crystals were then separated and the filtrate was used for ethanol recovery. The production of ethanol by using activated stable yeast crystals in conventional reactor is as follows.

TABLE 3

| S. NO. | PARAMETER | VALUES |
|---|---|---|
| 1. | Total volume (in liters) | 75.00 |
| 2. | Molasses used (Kg) | 21.00 |
| 3. | Initial specific gravity | 1.090 |
| 4. | Final specific gravity | 1.026 |
| 5. | Initial sugar concentration (%) | 14.90 |
| 6. | Sugar concentration in broth (%) | 2.15 |
| 7. | Alcohol produced (%) | 7.15 |
| 8. | Fermentation efficiency | 89.01 |
| 9. | Alcohol recovery per tone of molasses | 250.88 |
| 10. | Fermentation time | 30 |
| 11. | Rate of ethanol production | 8.36 |
| | (liters/hour/tone of molasses) | |

### EXAMPLE 4

### Ethanol Production Using Activated Yeast Crystals in Modified Conventional Reactor

The same experiment was performed as trial 2. The prepared activated stable yeast crystals and modified conventional fermentation vessel were used in this experiment. 75 liters of 1.090 specific gravity fermentation broth having initial sugar concentration of 14.90 (%/) was added into the modified conventional fermentation vessel. To this 1% (W/V) of activated yeast crystals were added. The fermentation was continued under constant mixing conditions in the modified fermentation vessel at 30° C. for 11 hours. The activated stable yeast crystals were then separated and the filtrate was used for ethanol recovery. The production of ethanol by using activated stable yeast crystals in modified conventional reactor is as follows.

TABLE 4

| S. NO. | PARAMETER | VALUES |
|---|---|---|
| 1. | Total volume (in liters) | 75.00 |
| 2. | Molasses used (Kg) | 21.00 |
| 3. | Initial specific gravity | 1.090 |
| 4. | Final specific gravity | 1.024 |
| 5. | Initial sugar concentration (%) | 14.90 |
| 6. | Sugar concentration in broth (%) | 2.10 |
| 7. | Alcohol produced (%) | 7.23 |
| 8. | Fermentation efficiency | 89.66 |
| 9. | Alcohol recovery per tone of molasses | 253.68 |
| 10. | Fermentation time | 11 |
| 11. | Rate of ethanol production | 23.06 |
| | (liters/hour/tone of molasses) | |

### EXAMPLE 5

### Ethanol Production Using Activated Yeast Crystals in Conventional Batch Reactor

The same experiment was performed as similar to example 3 using high gravity fermentation solution. The activated stable yeast crystals from the earlier experiments were reused. One hundred liters of fermentation broth having the specific gravity of 1.098 was prepared in conventional fermentation vessel by diluting the molasses with water and the initial sugar concentration in the fermentation broth was measured using conventional procedures. The 1% of activated stable yeast crystals were added to fermentation vessel. The fermentation was continued at 30° C. till the specific gravity of the fermentation broth was reached to 1.038. The activated stable yeast crystals were then separated and the filtrate was used for ethanol recovery. The production of ethanol by using activated stable yeast crystals in conventional reactor is as follows.

TABLE 5

| S. NO. | PARAMETER | VALUES |
|---|---|---|
| 1. | Total volume (in liters) | 100.00 |
| 2. | Molasses used (Kg) | 30.65 |
| 3. | Initial specific gravity | 1.098 |
| 4. | Final specific gravity | 1.038 |
| 5. | Initial sugar concentration (%) | 16.72 |
| 6. | Sugar concentration in broth (%) | 2.16 |
| 7. | Alcohol produced (%) | 8.52 |
| 8. | Fermentation efficiency | 92.88 |
| 9. | Alcohol recovery per tone of molasses | 266.25 |
| 10. | Fermentation time | 40 |
| 11. | Rate of ethanol production | 6.65 |
| | (liters/hour/tone of molasses) | |

### EXAMPLE 6

### Ethanol Production Using Activated Yeast Crystals in Modified Conventional Reactor

The same experiment was performed using high gravity fermentation broth. The prepared activated stable yeast crystals and modified conventional fermentation vessel were used in this experiment. One hundred liters of 1.098 specific gravity fermentation broth having initial sugar concentration of 16.71 (%) was added in the modified conventional fermentation vessel. To this 1% (W/V) of activated yeast crystals were added. The fermentation was continued under constant mixing conditions in the modified fermentation vessel at 30° C. till the specific gravity of the fermentation broth reached to 1.034. The activated stable yeast crystals were then separated and the filtrate was used for ethanol recovery. The production of ethanol by using activated stable yeast crystals in modified conventional reactor is as follows.

TABLE 6

| S. NO. | PARAMETER | VALUES |
|---|---|---|
| 1. | Total volume (in liters) | 100.00 |
| 2. | Molasses used (Kg) | 30.65 |
| 3. | Initial specific gravity | 1.098 |
| 4. | Final specific gravity | 1.034 |
| 5. | Initial sugar concentration (%) | 16.71 |
| 6. | Sugar concentration in broth (%) | 2.15 |
| 7. | Alcohol produced (%) | 8.62 |
| 8. | Fermentation efficiency | 93.97 |
| 9. | Alcohol recovery per tone of molasses | 269.38 |
| 10. | Fermentation time | 14 |
| 11. | Rate of ethanol production | 19.24 |
| | (liters/hour/tone of molasses) | |

### EXAMPLE 7

### Comparative Evaluation of Alcohol Fermentation Process Performed in Conventional and Modified Batch Reactor Using Activated Yeast Crystals

The ethanol fermentation performed in example 1 and 2 using activated yeast crystals in conventional and modified batch reactor were compared to evaluate the benefits of the present invention.

TABLE 7

| | | | IN |
|---|---|---|---|
| | | IN | MODIFIED |
| S. | | CONVENTIONAL | BATCH |
| NO. | PARAMETER | BATCH REACTOR | REACTOR |
| 1. | Total volume (in liters) | 100.00 | 100.00 |
| 2. | Molasses used (Kg) | 28.20 | 28.20 |
| 3. | Initial specific gravity | 1.090 | 1.090 |
| 4. | Final specific gravity | 1.024 | 1.024 |
| 5. | Initial sugar concentration (%) | 14.68 | 14.68 |
| 6. | Sugar concentration in | 1.86 | 1.86 |
| | broth (%) | | |
| 7. | Alcohol produced (%) | 6.90 | 7.04 |
| 8. | Fermentation efficiency | 85.43 | 87.10 |
| 9. | Alcohol recovery per tone of | 242.11 | 247.02 |
| | molasses | | |
| 10. | Fermentation time | 30 | 11 |
| 11. | Rate of ethanol production | 8.07 | 22.45 |
| | (liters/hour/tone of molasses) | | |

### EXAMPLE 8

### Comparative Evaluation of Alcohol Fermentation Process Performed in Conventional and Modified Batch Reactor Using Activated Yeast Crystals with High Density Fermentation Broth

The ethanol fermentation performed with high density fermentation broth (the specific gravity of the fermentation broth was more than 1.097) in example 5 and 6 using activated yeast crystals in conventional and modified batch reactor were compared to evaluate the importance of the present invention.

TABLE 8

| | | | IN |
|---|---|---|---|
| | | IN | MODIFIED |
| S. | | CONVENTIONAL | BATCH |
| NO. | PARAMETER | BATCH REACTOR | REACTOR |
| 1. | Total volume (in liters) | 100.00 | 100.00 |
| 2. | Molasses used (Kg) | 30.65 | 30.65 |
| 3. | Initial specific gravity | 1.098 | 1.098 |
| 4. | Final specific gravity | 1.038 | 1.034 |
| 5. | Initial sugar concentration (%) | 16.72 | 16.71 |
| 6. | Sugar concentration in | 2.16 | 2.15 |
| | broth (%) | | |
| 7. | Alcohol produced (%) | 8.52 | 8.62 |
| 8. | Fermentation efficiency | 92.88 | 93.97 |
| 9. | Alcohol recovery per tone of | 266.25 | 269.38 |
| | molasses | | |
| 10. | Fermentation time | 40 | 14 |
| 11. | Rate of ethanol production | 6.65 | 19.24 |
| | (liters/hour/tone of molasses) | | |

Advantages of the Invention

1. The main advantage of the present invention is reduction of batch fermentation time from 28-44 hours to 8-16 hours depending on the initial specific gravity of the fermentation broth.

2. The other advantage of the present invention is improvement of alcohol yield per tone to of molasses used during batch fermentation.

3. The another advantage of the present invention is improvement in rate of alcohol production.

4. Yet other advantage of the present invention is increasing of the plant capacity to the tune of more than 250% with minor modification of present basic fermentor structure.

5. Yet another advantage of the present invention is the reduction of product inhibition.

6. Still another advantage of the present invention is provision of cost effective alcohol fermentation process.

We claim

1. A process for production of ethanol using activated stable yeast crystals in a modified conventional batch reactor, said process comprising mixing stable yeast crystals to a low strength molasses solution having specific gravity in the range of 1.030 to 1.060 and incubating the stable yeast crystals therein to obtain activated stable yeast crystals, separating the activated stable yeast crystals, converting the low strength molasses solution into a molasses fermentation broth of a specific gravity in the range of 1.09 to 1.1 in a modified fermentation vessel fitting with a low speed mechanical stirrer, estimating the initial sugar concentration in the fermentation broth, adding the activated stable yeast crystals to the above fermentation broth in the range of 0.5 to 2.0% W/V and fermenting the mixture, terminating the fermentation when the specific gravity of the fermentation broth reaches a range of 1.014-1.045, separating the activated yeast crystals from fermented broth and recovering the ethanol from fermentation broth.

2. A process as claimed in claim 1 wherein the incubation of the stable yeast crystals in the is low strength molasses solution is carried out for a period of 4-48 hours at a temperature ranging between 24-36° C.

3. A process as claimed in claim 1 wherein the low strength molasses solution is obtained by mixing molasses with water.

4. A process as claimed in claim 1 wherein the fermentation is carried out for a time period in the range of 8-16 hours and at a temperature in the range of 28-40° C.

5. A process as claimed in claim 1 wherein the activated stable yeast crystals added in fermentation broth is preferably in the range of 0.5-1.5% (WV).

6. A process as claimed in claim 1 wherein the activated stable yeast crystals are separated from the molasses solution by draining out the solution or straining through a mesh or perforated bottoms.

7. A process as claimed in claim 1 wherein the low strength molasses solution of specific gravity in the range of 1.030 to 1.060 is prepared by mixing molasses and water by dilution or mechanical mixing.

8. A process as claimed in claim 1 wherein the mechanical stirrer has a speed in the range of 50-150 rpm.
